(19) United States
(12) Patent Application Publication
(10) Pub. No.: US 2005/0153303 A1
(43) Pub. Date: Jul. 14, 2005

(54) Method of mutagenic chain reaction

(75) Inventors: Marc Beauregard, Trois-Rivieres (CA); Sebastien Claveau, Trois-Rivieres (CA)

Correspondence Address: Marc Beauregard's, 2870 Bourjoly, Trois-Rivieres, QC, G8Z 2A9, CA

(73) Assignee: AGROTERRA BIOTECH INC., Trois-Rivieres (CA)

(21) Appl. No.: 10/776,180

(22) Filed: Feb. 12, 2004

Related U.S. Application Data
(60) Provisional application No. 60/446,518, filed on Feb. 12, 2003.

(51) Int. Cl. C12Q 1/68; C12P 19/34; C12N 15/01

(52) U.S. Cl. 435/6; 435/91.2; 435/441

(57) ABSTRACT

The present invention relates to a method for producing randomly mutated nucleic acid sequences and corresponding proteins or polypeptides. More particularly, the mutated nucleic acid sequences are generated by submitting a DNA template to polymerase chain reaction in a reaction buffer comprising an alcohol.

### BACKGROUND OF THE INVENTION

a) Field of the Invention

The present invention relates to the production of mutant proteins and peptides. More particularly, the present invention concerns a method for performing random-directed mutagenesis used in genetic engineering techniques. In one aspect, the method is exploited to generate mutated nucleic acid fragments encoding for mutant proteins with new or improved properties.

b) Description of Prior Art

During the last decade, spectacular advances have been reported in the field of genetic molecular evolution. Recently, several in vitro DNA recombination methods were developed, allowing applications such as mixing genetic material from a bank containing optimized sequence information, or construction of chimeric genes descending from related parental DNA molecules (Stemmer, (1994), Proc. Natl. Acad. Sci. USA, 91:10747-10751). However, these recombination methods require a biodiversity which is not always available in nature. In order to generate mutations, a variety of methods has been described, of which mutator bacteria strains (Cox, (1976), Annu. Rev. Genet. 10:135-156), chemical mutagenesis (Shortle, (1983), Methods Enzymol., 100:457-468), incorporation of nucleotides analogues (Mott et al., (1984) Nucleic Acids Res., 12:4139-4152), mutagenic oligonucleotides (Chiang L. et al., (1993), PCR Methods Applic., 2:210-217) and error-prone PCR (Leung, D. W. et al., (1989) Technique, 1:11-15). Among these, error-prone PCR (ep-PCR) is a method allowing easy and rapid generation of mutant banks.

Previous works concerning ep-PCR relied on Taq DNA polymerase due to its low inherent fidelity. Adding manganese ions to Taq polymerase PCR mixture was found to further decrease the enzyme polymerisation fidelity to a level that is suitable for random mutagenesis of genes.

It has been determined that error-prone PCR uses low-fidelity polymerization conditions to introduce a low level of point mutations randomly over a long sequence. In a mixture of fragments of unknown sequence, error-prone PCR can be used to induce mutagenesis in the mixture. This inability limits the practical application of error-prone PCR. Some computer simulations have suggested that point mutagenesis alone may often be too gradual to allow the large-scale block changes that are required for continued and dramatic sequence evolution. Further, it is known that error-prone PCR protocols do not allow for amplification of DNA fragments greater than 0.5 to 1.0 kb, limiting their practical application. In addition, repeated cycles of error-prone PCR can lead to an accumulation of neutral mutations with undesired results, such as affecting a protein's immunogenic properties but not its binding affinity.

In oligonucleotide-directed mutagenesis, a short sequence is replaced with a synthetically mutated oligonucleotide. This approach does not generate combinations of distant mutations and is thus not combinatorial. The limited library size relative to the vast sequence length means that many rounds of selection are unavoidable for protein optimization. Mutagenesis with synthetic oligonucleotides requires sequencing of individual clones after each selection round followed by grouping them into families, arbitrarily choosing a single family, and reducing it to a consensus motif. Such motif is resynthesized and reinserted into a single gene followed by additional selection. This step process constitutes a statistical bottleneck, is labor intensive, and is not practical for many rounds of mutagenesis.

Error-prone PCR and oligonucleotide-directed mutagenesis are thus useful for single cycles of sequence fine tuning, but rapidly become too limiting when they are applied for multiple cycles.

Another limitation of error-prone PCR is that the rate of down-mutations grows with the information content of the sequence. As the information content, library size, and mutagenesis rate increase, the balance of down-mutations to up-mutations will statistically prevent the selection of further improvements (statistical ceiling).

In cassette mutagenesis, a sequence block of a single template is typically replaced by a (partially) randomized sequence. Therefore, the maximum information content that can be obtained is statistically limited by the number of random sequences (i.e., library size). This eliminates other sequence families which are not currently best, but which may have greater long term potential.

Also, mutagenesis with synthetic oligonucleotides requires sequencing of individual clones after each selection round. Thus, such an approach is tedious and impractical for many rounds of mutagenesis. Some workers in the art have utilized an in vivo site specific recombination system to generate hybrids of combine light chain antibody genes with heavy chain antibody genes for expression in a phage system. However, their system relies on specific sites of recombination and is limited accordingly. Simultaneous mutagenesis of antibody CDR regions in single chain antibodies (scFv) by overlapping extension and PCR have been reported.

Different groups have described a method for generating a large population of multiple hybrids using random in vivo recombination. This method requires the recombination of two different libraries of plasmids, each library having a different selectable marker. The method is limited to a finite number of recombinations equal to the number of selectable markers existing, and produces a concomitant linear increase in the number of marker genes linked to the selected sequence(s).

According to the state of the art described above, it would be still advantageous to develop a method which allows for the production of large libraries of mutant DNA, RNA or proteins and the selection of particular mutants for a desired goal.

### SUMMARY OF INVENTION

One object of the present invention is to provide a method for inducing random mutations into a nucleic acid sequence comprising the steps of:
a) providing a nucleic acid sequence for use as DNA template:
b) submitting the DNA template to polymerization reaction with at least one DNA polymerase in presence of alcohol in concentration sufficient to lower the fidelity of the DNA polymerase and causing mutagenesis during the polymerization reaction.

The mutation can be a transversion, an insertion, a transition, or a deletion of at least one nucleotide.

The polymerization reaction can be performed as in the case of a polymerase chain reaction.

It will be evident to someone skilled in the art that the DNA polymerase can be a thermostable polymerase.

Alternatively, the DNA polymerase can be selected from the group consisting of polymerase produced by Thermus aquaticus, Thermococcus litoralis, Pyrococcus strain GB-D, Bacillus stearothermophilus, Pyrococcus furiosus, Bacteriophage T7 (type A or B), Thermus thermophilus, and Pyrococcus woesei, or can be a DNA polymerase of the type A or type B family polymerase.

The mutated nucleic acid sequence encodes for a biologically active protein.

The method of the invention may use an alcohol which is generally recognized as a chemical entity comprising a —OH group. The alcohol can be selected from the group consisting of propanol, ethanol, 2-aminoethanol, 1-propanol, 2-propanol, 1,2-propanediol, 1,3-propanediol, propanethiol, 1-butanol, 2-butanol, tert-butanol.

Another object of the present invention is to provide a method for preparing a library of mutated recombinant nucleic acid sequence comprising the steps of:
a) providing a nucleic acid sequence for use as DNA template; and
b) submitting the DNA template to polymerization with at least one DNA polymerase in presence of alcohol in concentration sufficient to lower the fidelity of the DNA polymerase and causing mutagenesis during the polymerization.

The method of the invention allows for the production of protein analogs that are biologically active protein analogs.

Again, the invention provides a method for producing a library of protein analogs comprising the steps of:
a) preparing a library of expression vectors, each expression vector comprising a mutated nucleic acid sequence prepared by the method of claim 1, operably linked to a promoter inducing transcription of the mutated nucleic acid sequence; and
b) allowing the expression vectors of step a) to produce a corresponding protein analogs.

Another object of the present invention is to provide the use of an alcohol in the preparation of a polymerization composition for inducing mutations in a DNA sequence.

In accordance with the present invention there is provided a polymerization composition for inducing mutations in a DNA sequence comprising a DNA polymerase and a sufficient amount of at least one alcohol for lowering the fidelity of the DNA polymerase during a process of polymerization.

It is another object of the present invention to provide a method for generating mutated polynucleotides encoding biologically active mutant polypeptides with enhanced, improved, or variant activities.

In another aspect of the invention, there is provided a method for producing biologically active mutant polypeptides encoded by randomly mutated polynucleotides. The present method allows for the identification of biologically active mutant polypeptides with enhanced biological activities.

For the purpose of the present invention the following terms are defined below.

The term “isolated” as used herein means that material is removed from its original environment (e.g., the natural environment if it is naturally occurring). For example, a naturally-occurring polynucleotide or polypeptide present in a living animal is not isolated, but the same polynucleotide or polypeptide separated from some or all of the coexisting materials in the natural system, is isolated.

The term “fidelity” refers to the error frequency of a given polynucleotide amplification reaction, e.g. a given set of PCR conditions. An example of an error frequency rate is the number of mutations that occur for every 1000 bp of synthesized PCR product.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

As representative examples of expression vectors which may be used there may be mentioned viral particles, baculovirus, phage, plasmids, phagemids, cosmids, fosmids, bacterial artificial chromosomes, viral DNA (e.g. vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, aspergillus and yeast) Thus, for example, the DNA may be included in any one of a variety of expression vectors for expressing a polypeptide. Such vectors include chromosomal, non-chromosomal and synthetic DNA sequences. Large numbers of suitable vectors are known to those of skill in the art, and are commercially available. The following vectors are provided by way of example; Bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used as long as they are replicable and viable in the host. Low copy number or high copy number vectors may be employed with the present invention. The term “amplification” means that the number of copies of a polynucleotide is increased.

The term “identical” or “identity” means that two nucleic acid sequences have the same sequence or a complementary sequence. Thus, “areas of identity” means that regions or areas of a polynucleotide or the overall polynucleotide are identical or complementary to areas of another polynucleotide or the polynucleotide.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence.

The term “related polynucleotides” means that regions or areas of the polynucleotides are identical and regions or areas of the polynucleotides are heterologous.

The term “library” as used herein means a collection of components such as polynucleotides, portions of polynucleotides or proteins. “Mixed library” means a collection of components which belong to the same family of nucleic acids or proteins (i.e., are related) but which differ in their sequence (i.e., are not identical) and hence in their biological activity.

The term “mutations” means changes in the sequence of a wild-type nucleic acid sequence or changes in the sequence of a peptide. Such mutations may be point mutations such as transitions or transversions. The mutations may be deletions or insertions.

### BRIEF DESCRIPTION OF THE DRAWINGS

FIG. 1 illustrates the DNA amplification by Vent™ (exo⁻) in standard condition and with different concentrations of 1-propanol;

FIG. 2 illustrates the base distribution in MB-1 His gene;

FIG. 3 illustrates the bias observed in the probability of a nucleotide being replaced, shown with different mutagenic conditions;

FIG. 4 illustrates the bias observed in the probability of a nucleotide being mutated for N (N=A, C, G or T) shown with different mutagenic conditions;

FIGS. 5a and 5b illustrate maximal length of amplification with different mutagenic PCR conditions; and

FIG. 6 illustrates mutation locations over the entire amplified DNA sequence.

### DESCRIPTION OF PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided a method for inducing mutagenesis in a nucleic acid or a corresponding amino acid sequence.

The subject invention provides methods for enzymatically producing primer extension products, e.g. in PCR applications, from template nucleic with at least one polymerase with a high error frequency, whereby high error frequency rate is meant an error frequency rate at or above, for example, 2 times 10⁻⁶, preferably at or above 4 times 10⁻⁶, and more preferably at or above 6 times 10⁻⁶ mutations per base pair per PCR cycle.

The polymerase chain reaction (PCR) in which nucleic acid primer extension product is enzymatically produced from template DNA are well known in the art, being described in U.S. Pat. Nos.: 4,683,202; 4,683,195; 4,800,159; 4,965,188 and 5,512,462, the disclosures of which are herein incorporated by reference.

In the subject methods, template nucleic acid is first contacted with primer and polymerase under conditions sufficient to enzymatically produce primer extension product. The nucleic acid that serves as template may be single stranded or double stranded, where the nucleic acid is typically deoxyribonucleic acid (DNA), where when the nucleic acid is single stranded, it will typically be converted to double stranded nucleic acid using one of a variety of methods known in the art. The length of the template nucleic acid may be as short as 50 bp, but usually be at least about 100 bp long, and more usually at least about 150 bp long, and may be as long as 10,000 bp or longer, but will usually not exceed 50,000 bp in length, and more usually will not exceed 20,000 bp in length. The nucleic acid may be free in solution, flanked at one or both ends with non-template nucleic acid, present in a vector, e.g. plasmid and the like, with the only criteria being that the nucleic acid be available for participation in the primer extension reaction. The template nucleic acid may be derived from a variety of different sources, depending on the application for which the PCR is being performed, where such sources include organisms that comprise nucleic acids, i.e. viruses; prokaryotes, e.g. bacteria; members of the kingdom fungi; and animals, including vertebrates, reptiles, fishes, birds, snakes, and mammals, e.g. rodents, primates, including humans, and the like. The nucleic acid may be used directly from its naturally occurring source and/or preprocessed in a number of different ways, as is known in the art. In some embodiments, the nucleic acid may be from a synthetic source.

The oligonucleotide primers with which the template nucleic acid (hereinafter referred to as template DNA for convenience) is contacted will be of sufficient length to provide for hybridization to complementary template DNA under annealing conditions (described in greater detail below) but will be of insufficient length to form stable hybrids with template DNA under polymerization conditions. The primers will generally be at least 10 bp in length, usually at least 15 bp in length and more usually at least 16 bp in length and may be as long as 30 bp in length or longer, where the length of the primers will generally range from 18 to 50 bp in length, usually from about 20 to 35 bp in length. The template DNA may be contacted with a single primer or a set of two primers, depending on whether linear or exponential amplification of the template DNA is desired. Where a single primer is employed, the primer will typically be complementary to one of the 3′ ends of the template DNA and when two primers are employed, the primers will typically be complementary to the two 3′ ends of the double stranded template DNA.

In the subject invention, unequal amounts of deoxyribonucleoside triphosphates (dNTPs) are employed. By unequal amounts is meant that at least one of the different types of dNTPs is present in the reaction mixture in an amount that differs from the amount at which the other dNTPs are present, i.e. a unique amount. The amount of difference will be at least about 1.5 and usually at least about 2. Usually the reaction mixture will comprise four different types of dNTPs corresponding to the four naturally occurring bases that are present, i.e. dATP, dTTP, dCTP and dGTP. Where the dNTPs employed are dATP, dTTP, dCTP and dGTP, only one of the dNTPs may be present at a unique amount, two of the dNTPs may be present at unique amounts, or all of the dNTPs may be present at unique amounts. In one preferred embodiment, dATP is present in a concentration greater than the individual concentrations of the remaining three dNTPs, i.e. dGTP, dCTP & dTTP. In another preferred embodiment, dGTP is present in a lower concentration than the individual concentrations of the remaining three dNTPs. In the subject methods, dATP can typically be present in an amount ranging from about 250 to 5000 μM, usually from about 300 to 1000 μM; dTTP can typically be present in an amount ranging from about 50 to 5000 μM, usually from about 100 to 400 μM; dCTP can typically be present in an amount ranging from about 50 to 5000 μM, usually from about 100 to 400 μM; and dGTP can typically be present in an amount ranging from about 10 to 150 μM, usually from about 20 to 100 μM.

Also present in the reaction mixtures of certain preferred embodiments of the subject invention is a melting point reducing agent, i.e. a reagent that reduces the melting point of DNA (or base-pair destabilization agent). Suitable melting point reducing agents are those agents that interfere with the hydrogen bonding interaction of two nucleotides, where representative base pair destabilization agents include: formamide, urea, thiourea, acetamide, methylurea, glycinamide, and the like, where urea is a preferred agent. The melting point reducing agent will typically be present in amounts ranging from about 20 to 500 mM, usually from about 50 to 200 mM and more usually from about 80 to 150 mM.

Following preparation of the reaction mixture, the reaction mixture is subjected to a plurality of reaction cycles, where each reaction cycle comprises: (1) a denaturation step, (2) an annealing step, and (3) a polymerization step. The number of reaction cycles can vary depending on the application being performed, but can usually be at least 15, more usually at least 20 and may be as high as 60 or higher, where the number of different cycles can typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture can usually be raised to, and maintained at, a temperature ranging from about 85 to 100° C., usually from about 90 to 98 and more usually from about 93 to 96° C. for a period of time ranging from about 3 to 120 sec, usually from about 5 to 30 sec.

Following denaturation, the reaction mixture can be subjected to conditions sufficient for primer annealing to template DNA present in the mixture. The temperature to which the reaction mixture is lowered to achieve these conditions can usually be chosen to provide optimal efficiency, and can generally range from about 50 to 75, usually from about 55 to 70 and more usually from about 60 to 68° C. Annealing conditions can be maintained for a period of time ranging from about 15 sec to 30 min, usually from about 30 sec to 5 min.

Following annealing of primer to template DNA or during annealing of primer to template DNA, the reaction mixture can be subjected to conditions sufficient to provide for polymerization of nucleotides to the primer ends in manner such that the primer is extended in a 5′ to 3′ direction using the DNA to which it is hybridized as a template, i.e. conditions sufficient for enzymatic production of primer extension product. To achieve polymerization conditions, the temperature of the reaction mixture can typically be raised to or maintained at a temperature ranging from about 65 to 75, usually from about 67 to 73° C. and maintained for a period of time ranging from about 15 sec to 20 min, usually from about 30 sec to 5 min.

The above cycles of denaturation, annealing and polymerization may be performed using an automated device, typically known as a thermal cycler. Thermal cyclers that may be employed are described in U.S. Pat. Nos. 5,612,473; 5,602,756; 5,538,871; and 5,475,610, the disclosures of which are herein incorporated by reference.

The subject polymerase chain reaction methods find use in any application where the production of enzymatically produced primer extension product from template DNA is desired, such as in the generation of specific sequences of cloned double-stranded DNA for use as probes, the generation of probes specific for uncloned genes by selective amplification of particular segments of cDNA or genomic DNA, the generation of libraries of cDNA from small amounts of mRNA, the generation of large amounts of DNA for sequencing, the analysis of mutations, generation of DNA fragments for gene expression, chromosome crawling, and the like. The subject methods find particular use in applications where low fidelity PCR is desired.

Also provided are kits for practicing the subject low fidelity PCR methods. The kits according to the present invention can comprise a polymerase and at least one of: (a) unequal amounts of dNTPs and (b) urea, where the polymerase may be a single polymerase or a combination of two or more different polymerases of type A or B. The subject kits may further comprise additional reagents which are required for or convenient and/or desirable to include in the reaction mixture prepared during the subject methods, where such reagents include an aqueous buffer medium (either prepared or present in its constituent components, where one or more of the components may be premixed or all of the components may be separate), and the like. The various reagent components of the kits may be present in separated containers, or may all be pre-combined into a reagent mixture for combination with template DNA. The subject kits may further comprise a set of instructions for practicing the subject methods.

In one embodiment of the present invention, there is provided a mutagenesis method performed by cyclic polymerization reaction using a DNA polymerase or mesophile enzyme. As described herein, a thermostable polymerase performs polymerization a given nucleic acid sequence at a relatively high temperature, while a Mesophile polymerase preferably carries out the polymerization at between about 25 to 45° C. For example, but not limited to, the enzyme can be a thermostable polymerase. It will be recognized from someone skilled in the art that the polymerase is preferably a DNA polymerase. The polymerases, which are generally well known by those skilled in the art, can be the Taq DNA polymerase originating from Thermus aquaticus. Other DNA polymerases that can be alternatively used to perform the method of the invention are, for example, naturally produced by Thermococcus litoralis, which produces a DNA polymerase of the type B family, and can be commercialized under the name Ventᵣ®, or Ventᵣ® (exo⁻). Other DNA polymerase used for the present invention can be selected from the group of polymerase produced by Pyrococcus species (GB-D Deep Ventᵣ® and Deep Ventᵣ® (exo⁻)), Bacillus stearothermophilus, Pyrococcus furiosus, Bacteriophage T7 (type A or B), Thermus thermophilus, and Pyrococcus woesei.

According to one embodiment of the present invention, the method provides peptides, proteins or polypeptides through which mutations confer different important characteristics, such as, but not limited to, resistance to high or low temperature, to proteases, to chemical agents such as organic solvents or denaturing agents, or to a pH that is normally adverse for non mutated proteins. Different other characteristics can be improved, such as for example the biological, biochemical or enzymatic activity, the affinity for a substrate or a ligand, the solubility, or as well as for improving the stability of the bi- or tri-dimensional conformation of the peptide or protein. The biophysical stability can also be improved with or without disulfide bridges or post-translational modifications.

Mesophile polymerase can alternatively be used as another embodiment to perform the method of the present invention. To perform PCR with a mesophile polymerase, the enzyme has to be added after each cycle of denaturation, once the thermal cycler has reach the proper temperature for DNA polymerization. This approach is necessary since mesophile polymerase are inactivated by the temperature required for DNA denaturation.

It will be recognized by those skilled in the art that different types of alcohols can be utilized to performed the present method of lowering the fidelity of a polymerase when working, therefore making it posible to obtain a desired level of mutation frequency in the polynucleotides and polypeptides. A targeted level of mutation is applied to a nucleotide or peptide sequence in order to obtain desired characteristic conferred or improved by the mutations. The level of mutation may vary significantly in a nucleotide sequence or a protein. It can be a percentage defined by the number of mutations as defined herein on the number of nucleic or amino acids. The level of mutation may be of about 0.01 to 25% in a DNA sequence or a protein depending on the needs.

The concentration of alcohol may vary from about 0.1 to 15%. Preferably, the concentration of alcohol in the reaction composition is between 1 to 8%. More preferably, the alcohol concentration in the reaction composition is of 7%. It will be recognized by someone skilled in the art that the reaction composition can be a buffer, a complete polymerization composition, or a part thereof, utilized in performing the mutating polymerization of a DNA fragment, such as a polymerase chain reaction, or a DNA polymerization carried out with the T7 DNA polymerase for example.

The present invention will be more readily understood by referring to the following examples which are given to illustrate the invention rather than to limit its scope.

### EXAMPLE I

### Random Mutated Polynucleotides And Polypeptides

In the present example, we demonstrated the lowering of the fidelity of a thermostable DNA polymerase during PCR was demonstrated by inducing a chemical stress using alcohol- and urea-water mixtures. It will be shown that alcohols can be used to alter polymerase performance at the level of: (i) mutation frequency, (ii) mutational bias, and (iii) maximal length of amplification. This is the first demonstration in the art of error-prone PCR using an alcohol in order to perform directed evolution experiments.

Materials & Methods

Template and Primers

The template used for PCR is the gene coding for MB-1 His (384 bp) (Grundy J. et al., (1998) Journal of Biotechnology, 63:9-15) cloned in the 6.6kb pMAL-c2 vector (New England Biolabs, cat no. M0257S). The template was obtained by plasmid purification of E. coli with XL-1 Blue (Stratagene) Miniprep protocol (Qiagen) and quantified using UV spectrometry.

PCR primer no. 1: 5′ATTCGAGCTCGAACAACAACAACAATAACAATAACAAC AACCTCGGGATCGAGGGAAGGATGGCTA-3′(SEQ ID NO:1) and primer no. 2: 5′GCCAAGCTTAGTGGTGGTGGTGGTGGTGAGCT-3′(SEQ ID NO:2) containing SacI and HindIII sites respectively (underscored letters) were purchased from Invitrogen life technologies and purified by PAGE.

PCR

Ventᵣ® (exo⁻) DNA polymerase and dNTPs were purchased from New England Biolabs. Manganese chloride and 1-propanol were purchased from Sigma Aldrich.

Control PCR condition: 2 units of Vent exo⁻DNA polymerase were used in a 50 μL reaction volume containing 200 μM of each dNTPs, 10mM KCl, 10mM (NH₄)₂SO₄, 2 mM tris-HCl pH 8.8, 2mM MgSO₄, 0.1% Triton™ X-100, 0.2 μM of each primers and 10ng of plasmid DNA. PCR were performed in a GeneAmp PCR system 9700 (Perkin-Elmer) as follows: 5 min at 95° C. (first denaturation), followed by 30 cycles of polymerisation [30 s at 95° C. (denaturation), 30 s at 65° C. (annealing) , 2 min at 72° C. (polymerisation)].

Manic PCR conditions: The buffer composition was the same as for the control PCR except that MnCI₂ and/or 1-propanol were added as described below. 1-propanol was added prior to other ingredients and aliquoted with a gastight Hamilton syringe to prevent inaccurate pipetting due to its fluidity. When a modified dNTPs ratio was used, fmal concentrations were 200 μM for dATP and dTTP and 800 μM for dCTP and dGTP.

PCR Measuring Maximal Length of Amplification

100ng of genomic DNA from Actinobacillus pleuropneumoniae was used as template. PCR cycles were as follows: 7 min at 95° C., then 30 cycles of polymerisation [45 s at 95° C., 45 s at 53° C., 1 min at 72°^{C}].

Cloning

pMAL-c2 vector (New England Biolabs) was digested with SacI and HindIII endonucleases (New England Biolabs) and gel purified (1% agarose) using Qiaex gel extraction kit (Qiagen) and Agarose A from LAB MAT. The 384 bp PCR products were also gel purified. This step was necessary since the primers auto-annealed each other and formed a 100 bp secondary product during PCR. The purified products were then concatenated in a kination/ligation using T4 Polynucleotide kinase and T4 DNA ligase (New England Biolabs) for 3 hours at room temperature followed by a restriction digestion using SacI and HindIII endonucleases for 5 hours at 37° C. Then, the digested PCR products were purified using PCR purification kit (Qiagen) and ligated with pMAL-c2 vector using T4 DNA ligase at 16° C. overnight.

Transformation

5 μL of ligation product mixed with 50 μL of E. coli XL-1 Blue hypercompetent cells were incubated on ice for 30 min, heatshocked at 42° C. for 45 sec and re-incubated on ice for 2 min. Then, 1mL of SOC medium was added and the mixture was incubated for 1 hour at 37° C. with shaking. Transformed Cells were then selected on LB agar plates containing 100 μg/mL ampicillin.

Determination of Mutation Rate

Plasmids from transformant colonies were purified with Qiaprep Spin miniprep kit (Qiagen) and digested with SacI/HindIII to confirm the presence of the 384 bp fragment corresponding to MB-1 His gene. Plasmids were then sequenced by the dye-terminator method (Service d'analyse et de synthèse, Université Laval). Complete sequences were analysed with LFASTA software (Chao K. M. (1998), Comput. Appl. Biosci., 8:481-487), allowing for comparison of control and mutated DNA. Mutation rate and mutation types were calculated from such a comparison.

Determination of Enzymatic Activity

Enzymatic activity of DNA polymerases has been determined by ethidium bromide staining of 1% agarose gel. Band intensity of 2 μL aliquots of amplicon issued from mutagenic PCR were compared to the intensity of bands obtained from several dilutions of standard PCR.

Results

Determination of Optimal Conditions For PCR

Three different types of mutation can occur during error-prone PCR: transition, transversion and insertion/deletion. Transition occurs when a purine is changed for the other purine (A→G), this also stands for pyrimidines, giving four possible transitions. Transversion occurs when a purine is replaced by a pyrimidine or vice versa (A→C), giving eight possible transversions. Insertion/deletion refers to a deoxynucleotide being incorporated/omitted during nucleic acid polymerisation. This results in a frame shift which causes undesired mutations such as non-sense codons.

In order to assess the impact of the chemical stress induced by alcohols and urea on PCR, we measured the amplification yield was measured and compared it to that of a standard PCR. The size of a library generated by error-prone PCR is proportional to the amplification yield, therefore it is of a paramount importance to maintain the amplification yield as high as possible. Concentration of alcohol leading to a reduction in amplification yield was detected were chosen to determine their impact on amplified sequences. Such concentrations were defined as “critical” concentration of alcohol. Critical concentrations of alcohols and urea determined with Taq, Ventᵣ® (exo−) and Deep Ventᵣ® (exo−) are summarized in Table 1.

TABLE 1
Critical concentrations of alcohols and urea determined with three
different DNA polymerases.

| | Taq | Ventᵣ ® (exo-) | Deep Ventᵣ ® (exo-) |
|---|---|---|---|
| | polymerase | polymerase | polymerase |
| | Critical concentration (% v/v) | | |
| Urea | 1.2 | 1.5 | 1.5 |
| | (0.20 M) | (0.25 M) | (0.25 M) |
| Isopropanol | N.A. | 10.0 | N.A. |
| Propanol | 2.5 | 8.0 | 8.0 |
| Butanol | 1.0 | 4.0 | 6.0 |

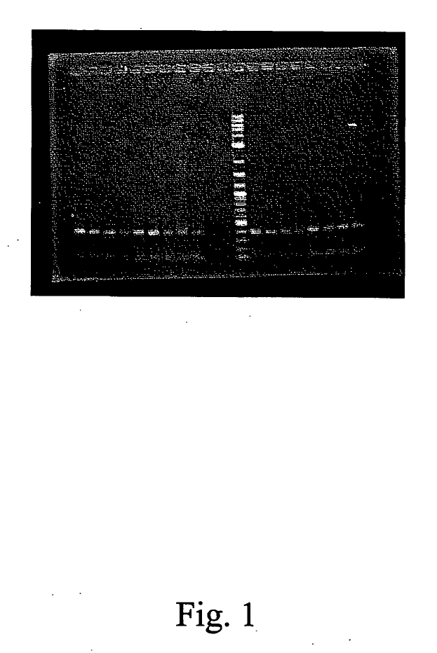

FIG. 1 shows the electrophoresis analysis of PCR products amplified by Ventᵣ® (exo−) under standard conditions and in the presence of different concentration of 1-propanol. The 400 bp bands correspond to MB-1 His gene and the 100 bp bands correspond to a secondary reaction product caused by the artefactual annealing of both primers. The different lanes are distributed as follows: wells 1 to 4 as control PCR dilutions corresponding to 100%, 75%, 50% and 25% of the normal amplification activity; wells 5 to 11 as PCR with 2.5%, 5.0%, 6.0%, 7.0% and 8.0%. 9.0% and 10.0% 1-propanol; well 12 as 2 Log DNA Ladder, from bottom to top: 100, 200, 300, 400, 500, 600, 700, 800, 900, 1000 bp; 1.2, 1.5, 2.0, 3.0, 4.0, 5.0, 6.0, 8.0 and 10.0kb; wells 13 to 16: as control PCR dilutions corresponding to 100%, 75%, 50% and 25% of the normal amplification activity; wells 17 to 20 as PCR with 5%, 6%, 7% and 8% 1-propanol.

There was no polymerisation activity in the presence of a concentration of 1-propanol above 8% ^{v}/ᵥ with Ventᵣ® (exo⁻) DNA polymerase. Consequently, we measured the mutation rate obtained when 7.0 and 8.0% 1-propanol were present during PCR.

A concentration of 7.0% propanol resulted in a mutation frequency of 0.27% without deletion. PCR with 8.0% propanol resulted in a mutation frequency of 0.58% and a single base deletion frequency corresponding to 0.048%, which is roughly ten times less frequent than substitution mutation.

We did not detect mutation in PCR using Taq or Ventᵣ® (exo−) in the presence of their respective critical concentration of urea. More than 2000 nucleotides were sequenced for these conditions.

We also tested the impact of manganese chloride (MnCl₂), a known mutagenic agent, alone and combined with 1-propanol on Ventᵣ® (exo⁻) DNA polymerase activity during PCR. The mutation rate obtained with 500 μM manganese alone was 3.7% without insertion or deletion.

The combination of both 1-propanol and MnCl₂ caused a diminution in enzymatic activity but the amount of PCR product was still suitable for cloning procedures. We tested the combination of 7.0% ^{v}/ᵥ 1-propanol with 250 and 500 μM MnCl₂. The resulting mutation rates were 1.50 and 2.30%, respectively.

Mutation types obtained with 1-propanol, manganese chloride and combination of both chemicals have been analysed separately assuming that each condition had its own mutagenic impact on the enzyme.

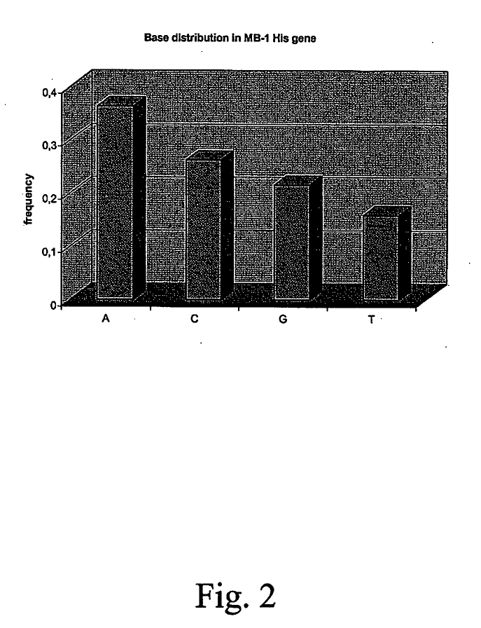

We expected a profile similar to the base content of MB-1 His gene (shown in FIG. 2) for the nucleotides to be mutated. However, the analysis of mutation types revealed a mutational bias observed with any of the chemical used, alone or in combination.

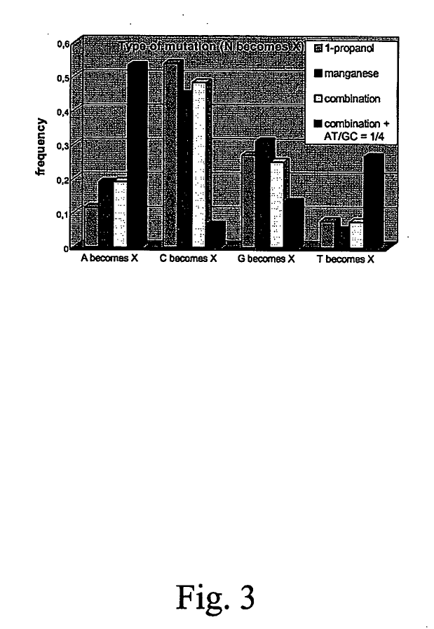

The mutation profile obtained with 7.0 or 8.0% propanol showed a trend favoring the mutation of guanines and cytosines, which represented 67% of total mutations. FIG. 3 shows a bias observed in the probability of a nucleotide being replaced, shown with different mutagenic conditions used in this work. On the X axis, in the title “A becomes X”, X is C, G or T, and so forth.

For Taq polymerase, the mutation profile obtained in the presence of 500 μM MnCl₂ showed a trend favouring the mutation of adenines and thymines, which represented 72% of total mutations. Here, we clearly observe that the mutation profile is dependant of the polymerase used in the mutagenic PCR.

We modified the dNTP ratio to change the mutational bias by as this is known to influence the type of mutation occurring as well as the mutation frequency (Cadwell and Joyce, (1992) PCR methods appl. 2:28-33; Vartanian et al., (1996), Nucleic Acids Res., 14:2627-2631). Sequencing data from PCR with 8.0% propanol and a ratio of AT/GC=¼ caused a modification in mutation profile lowering to 51% the frequency of apparition of adenines (A) and thymines (T). Moreover, mutation frequency and deletion frequency were enhanced to 0.98% and 0.13%, respectively.

The combination of propanol and manganese in a PCR using Ventᵣ® (exo−) polymerase reacted differently to a variation of nucleotide ratio. Using a ratio AT/GC=¼, the combination of 7.0% propanol and 0.5mM manganese chloride resulted in adenines and thymines being preferentially replaced, accounting for 81% of the mutations. Results are summarized in Table 2.

TABLE 2

| Mutation | Deletion | Maximal | | Amplification | Bias indicator | | |
|---|---|---|---|---|---|---|---|
| Frequency^{a} | Frequency^{b} | lenght of | Sequenced | yield | A, T | G, C | N turns |
| (%) | (%) | amplification | Nucleotides | (% of C+)^{c} | turns N^{d} | turns N | A, T^{e} |

Ventᵣ ® (exo-)

| | | | | | | | | |
|---|---|---|---|---|---|---|---|---|
| 7% propanol | | | | | | | | |
| Equimolar dNTPs | 0.27 | 0.00 | 2.8 kb | 2516 | 90 | 14% | 86% | 71% |
| AT/GC = 1/4 | 0.27 | 0.17 | 1.5 kb | 1152 | 50 | N.A. | N.A. | N.A. |
| 0.25 mM MnCl₂ | 1.82 | 0.18 | 0.8 kb | 3831 | 25 | 32% | 50% | 70% |
| 0.50 mM MnCl₂ | 2.30 | 0.00 | 0.8 kb | 1151 | 25 | 29% | 71% | 67% |
| 8% propanol | | | | | | | | |
| Equimolar dNTPs | 0.58 | 0.08 | 0.8 kb | 4127 | 75 | 25% | 67% | 67% |
| AT/GC = 1/4 | 0.98 | 0.13 | 0.8 kb | 4608 | 75 | 24% | 62% | 51% |
| 0.5 mM MnCl₂ | | | | | | | | |
| Equimolar dNTPs | 3.70 | 0.00 | 0.8 kb | 1533 | 75 | 23% | 77% | 83% |
| AT/GC = 1/4 | 0.52 | 0.00 | 1.6 kb | 3072 | 75 | 88% | 12% | 25% |
| AG/CT = 1/4 | 2.30 | 0.03 | 0.8 kb | 1151 | 75 | 30% | 67% | 73% |

Taq

| | | | | | | | | |
|---|---|---|---|---|---|---|---|---|
| 2.5% propanol | 0.13 | 0.04 | N.A. | 2250 | 50 | N.A. | | N.A. |
| 0.5 mM MnCl₂ | | | | | | | | |
| Equimolar dNTPs | 8.48 | 0.04 | 0.4 kb | 1687 | 75 | 72% | 24% | 43% |

^{a}Calculated as the number of mutation divided by the number of sequenced nucleotides, multiplied by 100.
^{b}Calculated as the number of deletion divided by the number of sequenced nucleotides multiplied by 100.
^{c}DNA yield of mutagenic PCR compared to standard PCR, estimated from band intensity after ethidium bromide staining of agarose gel.
^{d}Number of adenines and thymines mutated expressed in percentage of total mutations.
^{e}Number of nucleotide mutated for adenines and thymines expressed in percentage of total mutations.

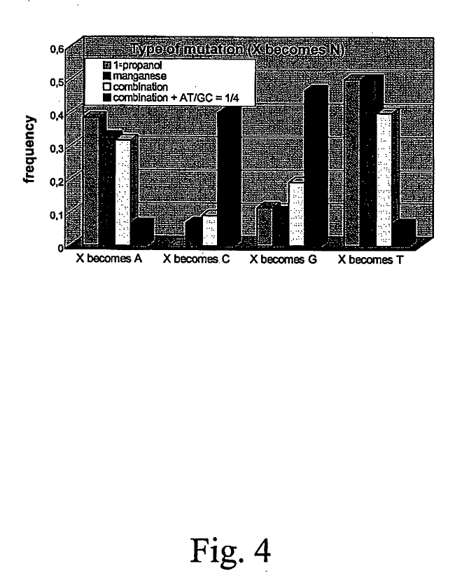

FIG. 4 shows a bias observed in the probability of a nucleotide being mutated for N (N=A, C, G or T) shown with different mutagenic conditions used in this work. For example, in X becomes A, X is C, G or T, and so forth.

Vent® exo− can amplify DNA molecules as long as 15kb under standard manufacturer condition (New England Biolabs Inc (2002-2003). Maximum length of amplification achieved with Vent® exo− was evaluated in a PCR allowing the amplification of DNA molecules of 0.8kb, 1.6kb and 2.8kb simultaneously. In presence of 7.0% propanol, the enzyme was able to amplify amplicons of 2.8kb. In presence of 8.0% propanol, the longest amplicon obtained was 0.7kb.

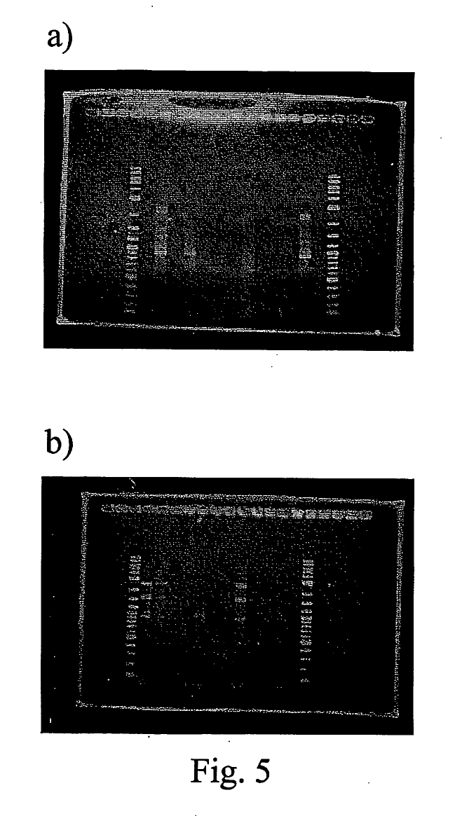

FIGS. 5a and 5b show the maximal length of amplification obtained with different mutagenic PCR conditions. In FIG. 5a defines wells 1 and 8 contain 2 Log DNA Ladder, from bottom to top: 100, 200, 300, 400, 500, 600, 700, 800, 900, 1000 bp; 1.2, 1.5, 2.0, 3.0, 4.0, 5.0, 6.0, 8.0 and 10.0kb; wells 2 and 7 contain standard PCR product; well 3 contains PCR with 500 μM MnCl₂; well 4 as show PCR with 500 μM MnCl₂+7% 1-propanol; well 5: PCR with ^{AT}/_{GC}=¼; and well 6 as: PCR with ^{AT}/_{GC}=¼+500 μM MnCl₂+7% 1-propanol. In FIG. 5b defines wells 1 and 14 contain 2 Log DNA Ladder; well 2 as: standard PCR product; well 3: as PCR with 7% 1-propanol; well 4: PCR with 8% 1-propanol; well 5: PCR with 7% 1-propanol+250 μM MnCl₂; well 6: PCR with 500 μM MnCl₂; wells 7 and 8 are empty; well 9: as standard PCR; well 10: PCR with ^{AT}/_{GC}=¼; well 11: PCR with ^{AT}/_{GC}=¼+7% 1-propanol; well 12: as PCR with ^{AT}/_{GC}=¼+500 μM MnCl₂; and well 13: PCR with ^{AT}/_{GC}=¼+7% 1-propanol+500 μM MnCl₂.

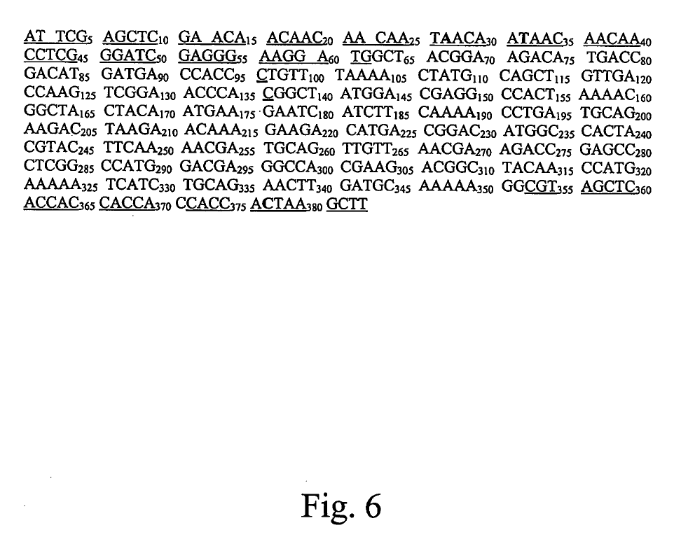

Analysis of mutation location revealed a random distribution throughout the gene sequence with few mutations located in primer regions, as shown in FIG. 6. where lower case letter indicate nucleotide mutated once, bold letter indicate nucleotide mutated twice and thick underscored letter indicate nucleotide mutated three times. Underlined regions correspond to oligonucleotide annealing sequences.

Taq DNA polymerase showed a low tolerance to 1-propanol; no polymerisation activity was detected above 2.5% ^{v}/ᵥ 1-propanol. PCR with 1.0 and 2.5% 1-propanol were done with Taq DNA polymerase. Of the 2250 nucleotides sequenced from Taq/1-propanol PCR, only 3 mutations have been found (0.13%). Considering the low mutation frequency obtained with Taq in presence of critical 1-propanol concentration, we did not further investigate this condition.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for inducing random mutations into a nucleic acid sequence comprising the steps of:
a) providing a nucleic acid sequence for use as DNA template:
b) submitting said DNA template to polymerization reaction with at least one DNA polymerase in presence of at least one alcohol in concentration sufficient to destabilize said DNA polymerase and causing mutagenesis during said polymerization reaction.

2. The method of claim 1, wherein said mutation is a transversion, an insertion, a transition, or a deletion of at least one nucleotide.

3. The method of claim 1, wherein said polymerization reaction is a polymerase chain reaction.

4. The method of claim 1, wherein said DNA polymerase is a thermostable or a mesophile polymerase.

5. The method of claim 1, wherein said DNA polymerase is selected from the group consisting of polymerase produced by Thermus aquaticus, Thermococcus litoralis, Pyrococcus species GB-D, Bacillus stearothermophilus, Pyrococcus furiosus, Bacteriophage T7 (type A or B), Thermus thermophilus, and Pyrococcus woesei.

6. The method of claim 1, wherein said DNA polymerase is a DNA polymerase of the type A or type B family polymerase.

7. The method of claim 1, wherein said mutated nucleic acid sequence encodes for a biologically active protein.

8. The method of claim 1, wherein said alcohol is a chemical entity comprising a —OH group.

9. The method of claim 1, wherein said alcohol is selected from the group consisting of propanol, ethanol, 2-aminoethanol, 1-propanol, 2-propanol, 1,2-propanediol, 1,3-propanediol, propanethiol, 1-butanol, 2-butanol, tert-butanol.

10. The method of claim 1, wherein said polymerization reaction is performed with a composition containing alcohol and nucleotides A, T, G, and C under conditions that allow for controlling mutational bias

11. A method for preparing a library of mutated recombinant nucleic acid sequence comprising the steps of:
a) providing a nucleic acid sequence for use as DNA template:
b) submitting said DNA template to polymerization with at least one DNA polymerase in presence of alcohol in concentration sufficient to lower the fidelity of said DNA polymerase and causing mutagenesis during said polymerization.

12. The method of claim 11, wherein said DNA polymerase is a thermostable polymerase.

13. The method of claim 11, wherein said protein analogs are biologically active protein analogs.

14. A method for producing a library of protein analogs comprising the steps of:
a) preparing a library of expression vectors, each expression vector comprising a mutated nucleic acid sequence prepared with the method of claim 1, operably linked to a promoter inducing transcription of said mutated nucleic acid sequence;
b) allowing said expression vectors of step a) to produce a corresponding protein analogs.

15. Use of an alcohol in the preparation of a polymerization composition for inducing mutations in a DNA sequence.

16. A polymerization composition for inducing mutations in a DNA fragment comprising a DNA polymerase and a sufficient amount of at least one alcohol for destabilizing said DNA polymerase during a process of polymerization.

17. A method for inducing mutations in a DNA fragment comprising adding alcohol in a polymerization reaction of a DNA template.


SEQUENCE LISTING (3 sequences)

SEQ ID NO: 1 - DNA, 66 bp, Artificial Sequence
  primer_bind (1)...(66): Sense oligo-primer
attcgagctcgaacaacaacaacaataacaataacaacaacctcgggatcgagggaaggatggcta

SEQ ID NO: 2 - DNA, 32 bp, Artificial Sequence
  primer_bind (1)...(32): antisense oligo-primer
gccaagcttagtggtggtggtggtggtgagct

SEQ ID NO: 3 - DNA, 384 bp, Artificial Sequence
  misc_recomb (1)...(384): Mutated MB-1
attcgagctcgaacaacaacaacaataacaataacaacaacctcgggatcgagggaaggatggctacggaagacatgaccgacatgatgaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtacttcaaaacgatgcagttgttaacgaagaccgagccctcggccatggacgaggccacgaagacggctacaaccatgaaaaatcatctgcagaacttgatgcaaaaaggcgtagctcaccaccaccaccaccactaagctt